(19) United States
(12) Patent Application Publication
(10) Pub. No.: US 2013/0317504 A1
(43) Pub. Date: Nov. 28, 2013

(54) Orthopedic Implants Having Improved Strength and Imaging Characteristics

(71) Applicants: David C. Paul, Phoenixville, PA (US); William S. Rhoda, Media, PA (US)

(72) Inventors: David C. Paul, Phoenixville, PA (US); William S. Rhoda, Media, PA (US)

(21) Appl. No.: 13/478,909

(22) Filed: May 23, 2012

(51) Int. Cl. A61B 17/68

(52) U.S. Cl. 606/77; 606/60; 606/76

(57) ABSTRACT

Orthopedic implants having improved strength and imaging characteristics are provided. The implants can comprise an inner core member that is encased at least in part by an outer encasing member. The inner core member can be formed of a first material that imparts improved strength to the implant, while the outer encasing member is formed of a second material that imparts improved imaging characteristics to the implant. Alternatively, the implants can include a single-piece member formed of a first material that is coated by a coating layer of a second material.

### FIELD OF THE INVENTION

The present invention is generally directed to orthopedic implants and in particular, orthopedic implants having improved strength and imaging characteristics.

### BACKGROUND OF THE INVENTION

Numerous procedures exist to alleviate pain caused by bone disease, trauma and fracture. To assist in treatment, a number of implants, such as bone screws and spacers, are used during surgical procedures. There is a continuing need for improved implants to ensure the safety of patients.

### SUMMARY OF THE INVENTION

Various embodiments of orthopedic implants are provided. In some embodiments, an orthopedic implant comprises an inner member comprising a shaft having a proximal end and a distal end, wherein the inner member is formed of a first material. The implant further comprises an outer member encasing at least a portion of the inner member, wherein the outer member is formed of a second material. The first material is of a greater strength than the second material.

In other embodiments, an orthopedic implant comprises an inner member comprising a shaft member having a proximal end and a distal end, wherein the shaft transitions into a head member, and wherein the inner member is formed of a first material. The implant further comprises an outer member that encases at least a portion of the inner member, wherein the outer member is formed independently from the inner member. The first material is ferromagnetic and the second material is non-ferromagnetic.

In other embodiments, an orthopedic implant comprises an inner member, wherein the inner member comprises a superior surface and an inferior surface and an opening formed through the superior surface and inferior surface. The opening is configured to receive graft material. The implant further comprises an outer member formed over at least a part of the inner member, wherein the outer member is configured to leave the graft hole exposed. The inner member is formed of a first material, while the outer member is formed of a second material, wherein the first material has a greater strength than the second material.

### BRIEF DESCRIPTION OF THE DRAWINGS

The invention will be more readily understood with reference to the embodiments thereof illustrated in the attached figures, in which:

FIG. 1 illustrates a multi-piece bone screw according to some embodiments.

FIG. 2 illustrates a single-piece bone screw according to some embodiments.

FIG. 3 illustrates a multi-piece, dual-diameter screw according to some embodiments.

FIG. 4 illustrates a single-piece dual-diameter screw according to some embodiments.

FIG. 5 illustrates a bone screw used in a bone fracture according to some embodiments.

FIG. 6 illustrates a bone screw used in a sacroiliac joint fusion procedure according to some embodiments.

FIG. 7 illustrates a bone plate according to some embodiments.

FIG. 8 illustrates an improved bone screw in use with the bone plate of FIG. 7 according to some embodiments.

FIG. 9 illustrates a multi-piece spacer according to some embodiments.

FIG. 10 illustrates a single-piece spacer according to some embodiments.

### DETAILED DESCRIPTION OF THE ILLUSTRATED EMBODIMENTS

Embodiments of the invention will now be described. The following detailed description of the invention is not intended to be illustrative of all embodiments. In describing embodiments of the present invention, specific terminology is employed for the sake of clarity. However, the invention is not intended to be limited to the specific terminology so selected. It is to be understood that each specific element includes all technical equivalents that operate in a similar manner to accomplish a similar purpose.

The present application describes orthopedic implants that have improved strength and imaging characteristics. In some embodiments, an implant comprises multiple pieces, wherein the implant comprises an inner core member encased in part by an outer encasement member. The core member can be formed of a first material, while the encasement member can be formed of a second material. In some embodiments, the first material provides improved strength to the implant, while the second material provides improved imaging capabilities to the implant. In other embodiments, an implant comprises a single-piece body, but includes a unique shield or coating layer to cover at least a part of the implant body. The implant body can be formed of a first material that increases the strength of the implant, while the coating can be formed of a second material that increases the imaging capabilities of the implant.

It has been found that specific processes and materials can be combined to form implants having optimal strength and imaging capabilities. For example, an improved fixation screw is provided that includes an inner core member formed of a strong biocompatible material such as cobalt-chrome or cobalt-chromium alloy and an outer encasement member formed of an imaging friendly material such as titanium or titanium alloy. The improved screw comprises a unique screw within a screw. Such implants would have both ideal strength and imaging capabilities, thereby vastly increasing the safety of patients using the implants.

Among the orthopedic implants described herein that have improved strength and imaging capabilities are fixation devices, including screws, fasteners and pins, formed of multiple materials. Such fixation devices are used in bone fractures and as screws (e.g., pedicle screws) to attach other implants in surgical procedures. It has been found that it can be difficult to have a bone screw that has the strength to withstand breakage and fatigue, as well as good imaging characteristics. The present application alleviates these concerns by providing unique bone screws that are configured to incorporate multiple materials with ease, whereby a first material can impart added strength to the implant, while a second material can impart added imaging characteristics to the implant. For example, the screw can include a core formed of cobalt-chrome or cobalt-chromium alloy for added strength, as well as an encasement formed of titanium or a titanium alloy for improved imaging capabilities.

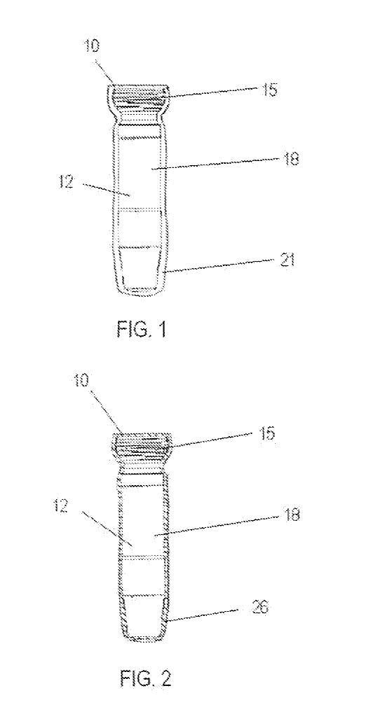

FIG. 1 illustrates a multi-piece bone screw having an inner core member covered by an outer encasement member. The inner core member 12 comprising a head 15 attached to a shaft 18. An outer encasement member 21 is provided over at least part of the inner core member 12, thereby forming a two-piece bone screw.

The inner core member 12 of the screw 10 includes a rounded head 15 that transitions into a shaft 18. In some embodiments, the head 15 includes a surface opening for receiving one or more instruments for guiding and implanting the screw 10.

Advantageously, in some embodiments, the inner core member 12 can be formed of a biocompatible material that will impart desirable strength to the implant. The material can be cobalt-chrome or cobalt-chromium alloys, which are advantageously strong and resistant to fatigue. Specific biocompatible materials can include but are not limited to cobalt-chrome-molybdenum (Co—Cr—Mo), cobalt-nickel-chromium-molybdenum (Co—Ni—Cr—Mo), stainless steel, tantalum, or other strong alloys. In some embodiments, the surface of the inner core member 12 can be surface hardened in order to enhance the strength of the material. In some embodiments, the inner core member 12 can have an ultimate tensile strength of at least 100,000 psi, or even 130,000 psi. In some embodiments, the ultimate tensile strength of the inner core member can fall between a range of 130,000 to 170,000 psi. In addition, in some embodiments, the inner core member 12 can have a fatigue limit of at least 10 million cycles at 610 MPa (90 ksi).

The implant further includes an outer encasement member 21 that covers at least a portion of the inner core member 12. In some embodiments, the outer encasement member 21 serves as a shell that encases a portion, but not all, of the inner core member 12. For example, the outer encasement member 21 can encase the shaft 18 of the inner core member 12, but leave the head 15 of the inner core member 12 exposed. In other embodiments, the outer encasement member 21 serves as a shell or case that encases the entire inner core member 12, including the head 15 and the shaft 18. In some embodiments, the outer encasement member 21 can include threads, while in other embodiments, the member 21 is non-threaded. The outer encasement member 21 can substantially mimic the contour of at least a portion of the inner core member 12, thereby providing a unique screw within a screw. In some embodiments, the outer encasement member 21 can be a shell casing that can be opened such that the inner core member 12 is inserted therein. In other embodiments, the outer encasement member 21 is molded over the inner core member 12. Numerous processes can be provided to form the outer encasement member 21. These processes include, but are not limited to, machining, die casting, forging, powder molding, beam melting, injection molding and laser forming.

Advantageously, in some embodiments, the outer encasement member 21 can be formed of a biocompatible material suitable for imaging, such as magnetic resonance imaging (MRIs). Such materials can include titanium or titanium alloys, which can also be corrosion-resistant. In some embodiments, the inner core member 12 is formed of a stronger material than the outer encasement member 21, thereby helping to counter failure and fatigue, while the outer encasement member 21 is more imaging friendly than the inner core member 12, thereby allowing for improved imaging in MRI and other imaging processes. For example, an inner core member of the screw 10 can be formed of cobalt-chrome or cobalt-chromium alloy, while an outer encasement member of the screw 10 can be formed of titanium or titanium alloy, thereby providing a unique implant having advantageous strength and imaging capabilities. In some embodiments, the outer encasement member 21 can have a tensile strength of 125,000 psi or less, or even 100,000 psi or less. Advantageously, in some embodiments, the tensile strength of the inner core member can be 1.2 to 1.4 times greater than the tensile strength of the outer encasement member. While the inner core member has greater strength than the outer encasement member, the outer encasement member can have greater imaging capabilities than the inner core member. For example, in some embodiments, the inner core member can be ferromagnetic, while the outer encasement member can be non-ferromagnetic, such that the outer member has greater imaging capabilities (e.g., in MRIs).

FIG. 2 illustrates an alternative bone screw having improved strength and imaging capabilities. In contrast to the bone screw in FIG. 1, which is formed of multiple-pieces, the bone screw 10 in FIG. 2 comprises a single-piece member 12 having an outer coating. The single-piece member 12 comprises a head 15 that transitions into a shaft 18. A coating layer 26 is provided over at least a portion of the single-piece member 12. In some embodiments, the material of the coating layer 26 is different from the material of the single-piece member 12. The single-piece member 12 can be formed of a strong, biocompatible material (e.g., cobalt-chrome or cobalt-chromium alloy), while the coating layer 26 is formed of an imaging friendly material, such as titanium or titanium alloy.

In some embodiments, the single-piece member 12 can be formed of a strong, biocompatible material. The member 12 can be coated with a coating layer 26. In some embodiments, the coating layer 26 is of a different material from the material of the body of the member 12. For example, in some embodiments, the single-piece member 12 can be formed of cobalt-chrome, while the coating layer 26 can be a layer of titanium. Advantageously, the single-piece member 12 can thus impart strength to the implant, while the coating layer 26 can impart beneficial imaging capabilities. In some embodiments, the coating layer 26 is comprised of titanium or a titanium alloy. Other non-limiting coating materials also include calcium phosphate, CaPO₄, calcium carbonate, CaCO₃, hydroxyapatite, and other metals and alloys. Various means can be used to apply the coating layer 26 to the member 12, including but not limited to dip coating, spray coating, plasma coating, flow coating, and vapor deposition processes. For example, in some embodiments, a titanium coating is applied to the single-piece member 12 via a dip coating process.

In some embodiments, the coating layer 26 covers the entire body of the single-piece member 12, including the head and shaft. In other embodiments, the coating layer 26 covers only a portion of the single-piece member 12. For example, in some embodiments, the coating layer 26 covers only the shaft and not the head of the single-piece member 12. In addition, in some embodiments, the coating can be applied sparingly, and in distinct patterns around the body of the member 12. For example, the coating can be applied in dotted marks intermittently around the body of the member 12. In addition, the coating can be applied in striated lines around the circumference of the member 12.

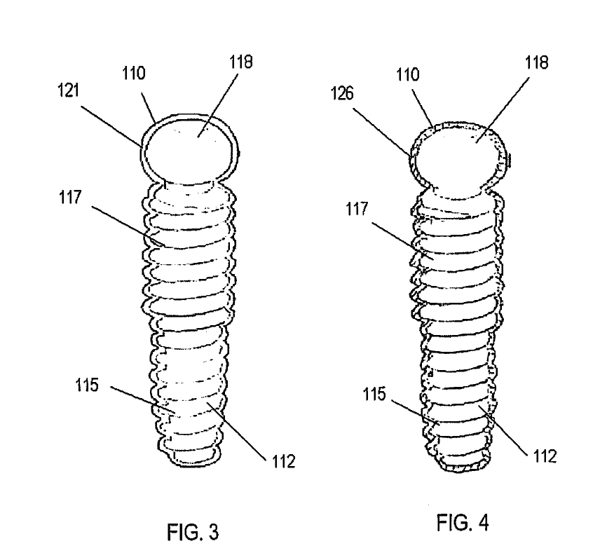

FIG. 3 illustrates a multi-piece, dual-diameter screw having improved strength and imaging characteristics. The screw 110 comprises an outer encasement member and an inner core member, thereby forming a screw within a screw. The inner core member 112 of the screw 110 comprises a first section 115 (e.g., a lead portion) having a first diameter and a second section 117 (e.g., a tail portion) having a second diameter. The advantage of providing a dual-diameter screw is that it can be used to form a secure fixation to bone which has regions with different characteristics such as dimensions and bone density, whereby the lead portion of the screw is received in a first region of the bone and the tail portion of the screw is received in a second region. As shown in the figure, the diameter of the first section 115 is less than the diameter of the second section 117. The screw 110 further includes a head portion 118 formed continuously with the second section 117. Advantageously, the inner core member 112 can be formed of a strong biocompatible material, including but not limited to cobalt-chrome, cobalt-chrome-molybdenum, cobalt-nickel-chromium-molybdenum, stainless steel, tantalum, and other strong alloys.

An outer encasement member 121 is formed over at least a portion of the inner core member 112. In some embodiments, the outer encasement member 121 is configured to encase the entire body of the inner core member 112. In some embodiments, the outer encasement member 121 is formed independently as a shell around the inner core member 112, whereby the inner core member 112 can be placed therein. In other embodiments, the outer encasement member 121 can be molded over the inner core member 112. In some embodiments, the outer encasement member 121 will have similar geometry of the inner core member 112, thereby advantageously maintaining the benefits of the dual diameter screw. Furthermore, the outer encasement member 121 and/or inner member 112 can include threads formed thereon. Any of the processes described above with forming outer encasement member 21 in FIG. 1 can also be applied herein.

In some embodiments, the outer encasement member 121 is formed of titanium or titanium alloys. In some embodiments, the outer encasement member 121 is of a different material from the inner core member 112. In some embodiments, the inner core member 112 can be formed of a material having a greater amount of strength (e.g., tensile strength) than the material of the outer encasement member 121, while the outer encasement member 121 can provide enhanced imaging capabilities to the implant. In some embodiments, the inner core member 112 of the dual diameter screw can be formed of cobalt-chrome or a cobalt-chromium alloy in order to enhance the strength of the screw, while the outer member 121 of the dual diameter screw can be formed of titanium or a titanium alloy to enhance the imaging properties of the screw.

FIG. 4 illustrates a dual-diameter screw having improved strength and imaging capabilities. The screw 110 comprises a single-piece member 112 with an outer coating formed thereover. The single-piece member 112 includes a first section 115 (e.g., a lead portion) with a first diameter and a second section 17 (e.g., a tail portion) having a second diameter, whereby the second diameter is greater than the first diameter. A coating layer 126 is formed over at least a part of the single-piece member 112. The coating layer 126 can be formed of any of the processes described above, including dip coating and plasma spraying.

In some embodiments, the body of the single-piece member 112 is of a different material from the coating layer 126. For example, in some embodiments, the single-piece member 112 can be formed of cobalt-chrome, while the coating layer 126 can be titanium or a titanium alloy. While in some embodiments, the coating layer 126 is applied substantially to the entire body of the member 112, in other embodiments, the coating layer 126 is applied discontinuously at select parts of the member 112. For example, the coating layer 126 can be applied as a spiral or helix around the dual diameter screw body, or can be applied as dots formed intermittently around the circumference of the screw.

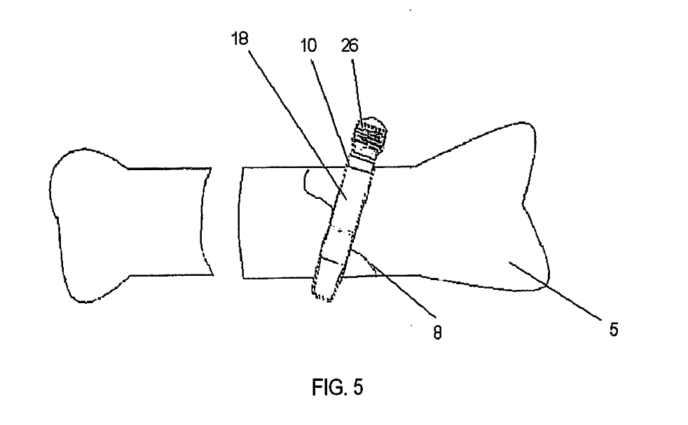

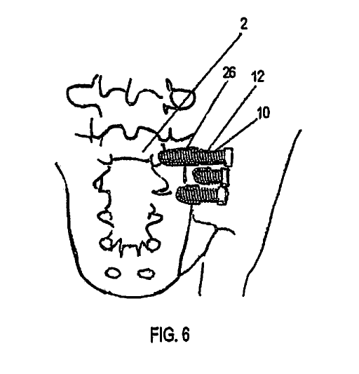

The screws described above can be used in various procedures. For example, FIG. 5 illustrates a coated bone screw used in a bone fracture, while FIG. 6 illustrates a coated bone screw used in a sacroiliac joint fusion procedure.

In FIG. 5, a screw 10 having improved strength and imaging characteristics is inserted into bone 5 to assist in treatment of fracture 8. The screw 10 includes a shaft 18, at least some of which is covered in a metallic coating 26. The shaft 18 can be of a different material from the metallic coating. In some embodiments, the shaft 18 is formed of cobalt-chrome, while the metallic coating 26 is formed of titanium. By having a body formed of cobalt-chrome, this advantageously reduces the risk of the screw breaking during or after implantation, while having a titanium coating improves the imaging properties of the implant.

In FIG. 6, a plurality of screws 10 having improved strength and imaging characteristic are inserted into a sacro-iliac joint (SI_joint) in order to assist in a fusion process. Each of the screws 10 includes a shaft coated with a metallic coating 26. The coating 26 is applied over only a portion of the shaft of each of the screws. In some embodiments, the body of the screws can be formed of a different material from the coatings, as discussed above. Moreover, in some embodiments, multiple screws can be coated in different areas in order to provide imaging capabilities in select areas.

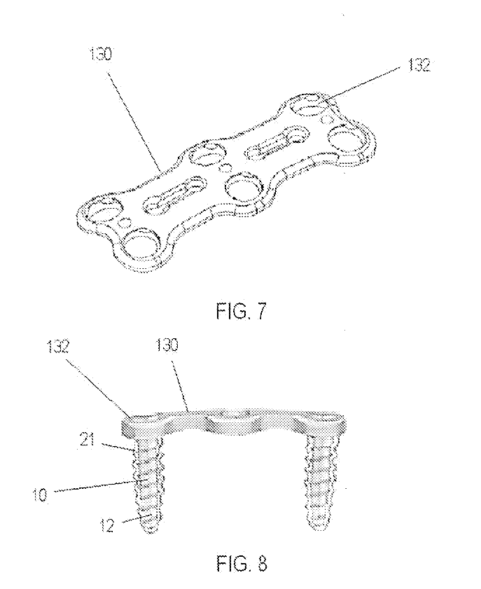

In FIG. 7, a bone plate for assisting in a fusion procedure is shown. The bone plate 130 comprises a number of openings 132 that can receive one or more improved bone screws as discussed above. The bone plate 130 is configured to extend across one or more vertebral bodies in the cervical, thoracic and/or lumbar regions to stabilize the vertebral bodies.

FIG. 8 illustrates the bone plate in FIG. 7 with improved bone screws received therein. The bone screws 10 are configured to have improved strength and imaging characteristics and include an inner core member 10 and an outer encasement member 21 formed thereover. The bone screws 10 are inserted into the bone plate 130 via the openings 132, and can be inserted into one or more vertebral bodies.

Among the orthopedic implants described herein are spacers formed of multiple materials. Spacers, which are inserted into an intervertebral disc space, are load-bearing devices that can also suffer from fatigue failure. In addition, it can be difficult to produce an image of the spacers within the patient. To alleviate these problems, the present application provides improved spacers that have improved strength and imaging characteristics. The spacers are formed of multiple materials, whereby a first part of the spacer is formed of a first material imparting improved strength, while a second part of the spacer is formed of a second material imparting improved imaging characteristics. The first part of the spacer can be formed of a strong material, such as cobalt-chrome, while the second part of the spacer can be formed of a different material, such as titanium.

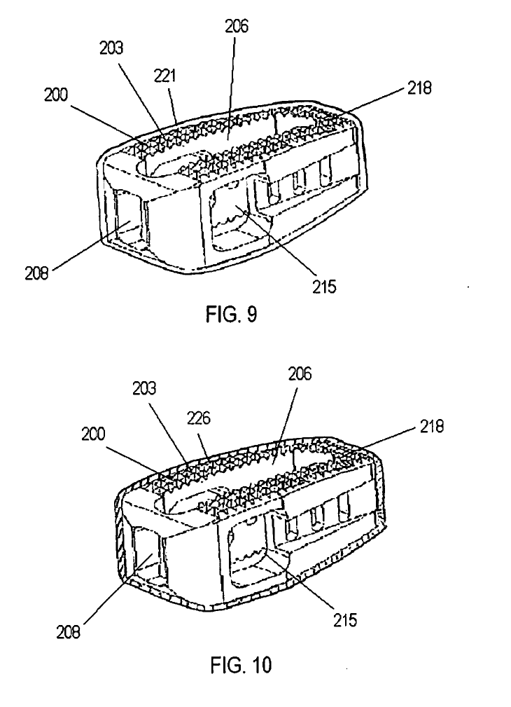

FIG. 9 illustrates a multi-piece spacer having improved strength and imaging characteristics. The spacer 200 comprises an inner core member 203 encased in part by an outer encasing member 221. The inner core member 203 includes a superior surface and an inferior surface, and an opening 206 that extends through the superior surface and inferior surface. The spacer 200 further includes side windows 215 and an opening 208 for receiving an insertion instrument. Natural and/or synthetic bone graft material can be inserted through the opening 206. Surface protrusions 218, such as teeth or ribbing, are formed on the superior and/or inferior surfaces to assist in gripping of adjacent vertebrae.

As shown in FIG. 9, the spacer 200 can comprise a substantially wedge-shaped member, although one skilled in the art will appreciate that the geometry is not so limited. For example, in some embodiments, the spacer can include an anterior surface that is concave and an opposing posterior surface that is convex. In some embodiments, the superior surface and inferior surface are substantially parallel, while in other embodiments, one is angled relative to the other to form a lordotic implant.

An outer encasing member 221 is formed around at least portions of the inner core member 203. In some embodiments, the outer encasing member 221 only covers portions of the inner core member 203, leaving other portions, such as the surface protrusions, windows and openings exposed. In some embodiments, the outer encasing member 221 comprises a case through which the inner core member 203 can be inserted, while in other embodiments, the outer encasing member 221 is molded or formed around the inner member. In some embodiments, the spacer inner core member 203 can be formed of a first material, while the outer encasing member 221 is formed of a second material. In some embodiments, the spacer inner core member 203 is composed of a first material such as cobalt-chrome for load-bearing strength, while the outer encasing member 221 is composed of a second material such as titanium for improved imaging capabilities. In other embodiments, the spacer inner core member 203 is formed of a non-metallic material, such as allograft bone. Furthermore, other materials, such as those discussed above with respect to the inner and outer members of the bone screw, can also be applied to the spacer.

FIG. 8 illustrates an alternative spacer having improved strength and imaging capabilities. In contrast to the prior spacer, however, the present spacer comprises an inner body 203 that is covered by an outer coating layer. The spacer 200 shares similar features to the spacer in FIG. 7, and includes an inner core member or body 203 with superior and inferior surfaces, surface protrusions 218, opening 206, windows 215 and instrument opening 208. However, rather than having an inner member and an outer member, the present spacer 200 has a coating layer 226 that is formed over at least some portions of the inner core member 203. In some embodiments, the inner core member 203 is composed of a first material such as cobalt-chrome for load-bearing strength, while the coating layer 226 is composed of a second material such as a titanium mixture for improved imaging capabilities. In some embodiments, the spacer is carefully inserted into a dip coating to coat portions of the spacer with titanium.

While the invention herein disclosed has been described by means of specific embodiments and applications thereof, numerous modifications and variations can be made thereto by those skilled in the art without departing from the scope of the invention.

What is claimed is:

1. An orthopedic implant comprising:
an inner member comprising a shaft having a proximal end and a distal end, wherein the inner member is formed of a first material; and
an outer member encasing at least a portion of the inner member, wherein the outer member is formed of a second material,
wherein the first material is of a greater strength than the second material.

2. The implant of claim 1, wherein the first material comprises cobalt-chrome.

3. The implant of claim 2, wherein the second material comprises titanium.

4. The implant of claim 1, wherein the outer member is independently formed from the inner member, and substantially conforms to at least a portion of the inner member.

5. The implant of claim 1, wherein the shaft includes a first portion having a first diameter and a second portion having a second diameter, wherein the second diameter is greater than the first diameter.

6. The implant of claim 1, wherein the outer member is molded onto the inner member.

7. An orthopedic implant comprising:
an inner member comprising a shaft member having a proximal end and a distal end, wherein the shaft transitions into a head member, and wherein the inner member is formed of a first material; and
an outer member that encases at least a portion of the inner member, wherein the outer member is formed independently from the inner member,
wherein the first material is ferromagnetic and the second material is non-ferromagnetic.

8. The implant of claim 7, wherein the first material of the inner member has a tensile strength of at least 1.2 times the tensile strength of the second material.

9. The implant of claim 7, wherein the shaft member includes a first portion having a first diameter and a second portion having a second diameter different from the first portion.

10. The implant of claim 7, wherein the shaft member has a constant radius from a proximal end to a distal end.

11. The implant of claim 7, wherein the first material comprises a cobalt alloy, while the second material comprises a titanium alloy.

12. The implant of claim 7, wherein the outer member includes threads formed thereon.

13. An orthopedic implant comprising:
an inner member, wherein the inner member comprises a superior surface and an inferior surface and an opening formed through the superior surface and inferior surface, wherein the opening is configured to receive graft material, the inner member being formed of a first material; and
an outer member formed over at least a part of the inner member, wherein the outer member is configured to leave the graft hole exposed, the outer member being formed of a second material,
wherein the first material has a greater strength than the second material.

14. The implant of claim 13, wherein the superior surface and inferior surface include surface protrusions in the form of teeth.

15. The implant of claim 13, wherein the implant further comprises a body having a concave surface opposed to a convex surface.

16. The implant of claim 13, wherein the second material comprises titanium.

17. The implant of claim 13, wherein the inner member has a greater tensile strength than the outer member.

18. The implant of claim 13, wherein the implant further comprises side windows.

19. The implant of claim 13, wherein the second material is non-ferromagnetic.

20. The implant of claim 13, wherein the second material comprises a titanium alloy.
